(19) United States
(12) Patent Application Publication
(10) Pub. No.: US 2025/0359786 A1
(43) Pub. Date: Nov. 27, 2025

(54) REGIONAL OXIMETRY POD

(71) Applicant: MASIMO CORPORATION, Irvine, CA (US)

(72) Inventors: Ammar Al-Ali, San Juan Capistrano, CA (US); Kevin Forrest, Rancho Santa Margarita, CA (US); David Dalke, Rancho Santa Margarita, CA (US); Walter M. Weber, Laguna Hills, CA (US)

(21) Appl. No.: 19/233,998

(22) Filed: Jun. 10, 2025

Related U.S. Application Data
(63) Continuation of application No. 18/334,969, filed on Jun. 14, 2023, now Pat. No. 12,357,203.
Application No. 18/334,969 is a continuation of application No. 17/039,456, filed on Sep. 30, 2020, now Pat. No. 11,717,194.
Application No. 17/039,456 is a continuation of application No. 15/801,257, filed on Nov. 1, 2017, now Pat. No. 10,799,160.
Application No. 15/801,257 is a division of application No. 14/507,639, filed on Oct. 6, 2014, now Pat. No. 9,839,379.
(60) Provisional application No. 61/887,878, filed on Oct. 7, 2013.
(60) Provisional application No. 61/887,856, filed on Oct. 7, 2013.
(60) Provisional application No. 61/887,883, filed on Oct. 7, 2013.
(60) Provisional application No. 62/012,170, filed on Jun. 13, 2014.

(51) Int. Cl. A61B 5/145; A61B 5/00; A61B 5/1455; H01R 13/52

(52) U.S. Cl. CPC A61B 5/14542; A61B 5/1455; A61B 5/14552; A61B 5/14553; A61B 5/6833; A61B 5/7275; A61B 5/742; A61B 5/746; A61B 5/14551; A61B 5/14557; A61B 2562/22; A61B 2562/222; A61B 2562/225; A61B 2562/227; A61B 2562/228; H01R 13/5224; H01R 2201/12

(57) ABSTRACT

A regional oximetry pod drives optical emitters on regional oximetry sensors and receives the corresponding detector signals in response. The sensor pod has a dual sensor connector configured to physically attach and electrically connect one or two regional oximetry sensors. The pod housing has a first housing end and a second housing end. The dual sensor connector is disposed proximate the first housing end. The housing at least partially encloses the dual sensor connector. A monitor connector is disposed proximate a second housing end. An analog board is disposed within the pod housing and is in communications with the dual sensor connector. A digital board is disposed within the pod housing in communications with the monitor connector.

### PRIORITY CLAIM TO RELATED PROVISIONAL APPLICATIONS

Any and all applications for which a foreign or domestic priority claim is identified in the Application Data Sheet as filed with the present application are hereby incorporated by reference under 37 CFR 1.57. The present application is a continuation of U.S. patent application Ser. No. 18/334,969, filed Jun. 14, 2023, which is a continuation of U.S. patent application Ser. No. 17/039,456, filed Sep. 30, 2020, which is a continuation of U.S. patent application Ser. No. 15/801,257, filed Nov. 1, 2017, which is a divisional of U.S. patent application Ser. No. 14/507,639, filed Oct. 6, 2014, titled Regional Oximetry Pod, which claims priority benefit under 35 U.S.C. § 119 (e) to U.S. Provisional Patent Application Ser. No. 62/012,170, filed Jun. 13, 2014, titled Peel-Off Resistant Regional Oximetry Sensor; U.S. Provisional Patent Application Ser. No. 61/887,878 filed Oct. 7, 2013, titled Regional Oximetry Pod; U.S. Provisional Patent Application Ser. No. 61/887,856 filed Oct. 7, 2013, titled Regional Oximetry Sensor; and U.S. Provisional Patent Application Ser. No. 61/887,883 filed Oct. 7, 2013, titled Regional Oximetry User Interface; all of the above-referenced provisional patent applications are hereby incorporated in their entireties by reference herein.

### BACKGROUND

Pulse oximetry is a widely accepted noninvasive procedure for measuring the oxygen saturation level of arterial blood, an indicator of a person's oxygen supply. A typical pulse oximetry system utilizes an optical sensor attached to a fingertip to measure the relative volume of oxygenated hemoglobin in pulsatile arterial blood flowing within the fingertip. Oxygen saturation (SpO₂), pulse rate and a plethysmograph waveform, which is a visualization of pulsatile blood flow over time, are displayed on a monitor accordingly.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, California and are incorporated in their entireties by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated in their entireties by reference herein. Advanced pulse oximetry systems including Masimo SET® low noise optical sensors and read through motion pulse oximetry monitors for measuring SpO₂, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad-8®, Rad-50, Rad®-5v or SatShare® monitors.

Advanced blood parameter measurement systems are described in at least U.S. Pat. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. Pub. No. 2006/0211925, filed Mar. 1, 2006, titled Physiological Parameter Confidence Measure and U.S. Pat. Pub. No. 2006/0238358, filed Mar. 1, 2006, titled Noninvasive Multi-Parameter Patient Monitor, all assigned to Cercacor Laboratories, Inc., Irvine, CA (Cercacor) and all incorporated in their entireties by reference herein. Advanced blood parameter measurement systems include Masimo Rainbow® SET, which provides measurements in addition to SpO₂, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad-87™ and Rad-57™ monitors, all available from Masimo. Such advanced pulse oximeters, low noise sensors and advanced blood parameter systems have gained rapid acceptance in a wide variety of medical applications, including surgical wards, intensive care and neonatal units, general wards, home care, physical training, and virtually all types of monitoring scenarios.

### SUMMARY

Regional oximetry, also referred to as tissue oximetry and cerebral oximetry, enables the continuous assessment of tissue oxygenation beneath a regional oximetry optical sensor. Regional oximetry helps clinicians detect regional hypoxemia that pulse oximetry alone can miss. In addition, the pulse oximetry capability in regional oximetry sensors can automate a differential analysis of regional to central oxygen saturation. Regional oximetry monitoring is as simple as applying regional oximetry sensors to any of various body sites including the forehead, forearms, chest, upper thigh, upper calf or calf, to name a few. Up to four sensors are connected to a conventional patient monitor via one or two regional oximetry pods. The pods advantageously drive the sensor optics, receive the detected optical signals, perform signal processing on the detected signals to derive regional oximetry parameters and communicate those parameters to a conventional patient monitor through, for example, standard USB ports.

One aspect of a regional oximetry pod drives the optical emitters of one or two regional oximetry sensors and receives the corresponding detector signals in response. The sensor pod has a dual sensor connector configured to physically attach and electrically connect one or two regional oximetry sensors. The pod housing has a first housing end and a second housing end. The dual sensor connector is disposed proximate the first housing end. The housing at least partially encloses the dual sensor connector. A monitor connector disposed proximate a second housing end. An analog board is disposed within the pod housing in communications with the dual sensor connector, and a digital board is disposed within the pod housing in communications with the monitor connector.

In various embodiments, the dual sensor connector has a pair of pod cables partially disposed within the pod housing. A first end of the pod cables is electrically connected to and mechanically attached to the analog board. A second end of the pod cables extends from the pod housing and terminates at a pair of sensor connectors. The sensor connectors are configured to physically attach and electrically connect up to two regional oximetry sensors. The dual sensor has a socket block at least partially disposed within the pod housing, and the socket block has socket contacts configured to electrically connect to a pair of regional oximetry sensors. The socket contacts are in electrical communications with the analog board. The monitor connector has a pod cable extending from the digital board and terminates at a monitor connector. The analog board has an analog board connector disposed on the analog board surface. The digital board has a digital board connector disposed on the digital board surface, and the analog board connector is physically and electrically connected to the digital board connector.

In further embodiments, the analog board mounts emitter drivers that activate the regional oximetry sensor emitters, the analog board has detector amplifiers that receive sensor signals from the regional oximetry detectors, and the analog board digitizes the sensor signals. The digital board has a digital signal processor (DSP) that inputs the digitized sensor signals. The DSP derives regional oximetry parameters from the sensor signals, and the regional oximetry parameters are communicated to a patient monitor via the pod cable and the monitor connector.

Another aspect of a regional oximetry pod is defining a pod having a first pod end and a second pod end, disposing a signal processor within the pod, extending a sensor connector from the first pod end and extending a monitor connector from the second pod end. Sensor signals are received from the first pod end. Signal processing on the sensor signals calculates a regional oximetry parameter, and the parameter is transmitted to the monitor connector for display on a standard patient monitor.

In various embodiments, disposing a signal processor within the pod comprises stacking an analog board to a digital board, extending a sensor cable from the analog board to the sensor connector and extending a monitor cable from the digital board to the monitor connector. This also comprises mounting and electrically connecting a DSP to the digital board and calculating the regional oximetry parameter within the DSP. This also comprises driving sensor emitters and receiving detector signals on the analog board, wherein extending a monitor connector includes attaching a monitor cable first end to the signal processor and attaching the monitor connector to a monitor cable second end. Extending a sensor connector comprises extending sensor connector cables from the first pod end, and attaching the sensor connector to the sensor connector cable distal the pod. Extending a sensor connector comprises attaching a socket block partially within the pod at the first pod end.

An additional aspect of a regional oximetry pod is a driver means for transmitting a drive signal to a plurality of emitters, and an amplifier means for receiving a response signal from at least one detector in optical communications with the emitters. A dual connector means is for communicating the drive signal and the response signal to the drive means and the amplifier means. A housing means is for enclosing the driver means and the amplifier means and for at least partially enclosing the dual connector means. An analysis means is for deriving physiological parameters from the response signal, and a monitoring means is for communicating the physiological parameters to a display. The driver means and the amplifier means comprise an analog board means disposed within the housing means.

For various embodiments, the analysis means comprises a digital board means disposed within the housing means. Board connectors interconnect the analog board means and the digital board means. A frame means is for mechanically stabilizing the analog board means connected to the digital board means. The dual connector means has connector cables extending from the housing means between the analog board means and a plurality of sensor connectors. The dual connector means has a socket block partially disposed within the housing means and is configured to receive dual sensor plugs. A monitoring means comprises a pod cable extending from the digital board means and the housing means and terminating at a USB connector.

### BRIEF DESCRIPTION OF THE DRAWINGS

FIG. 1 is a general block diagram of a pod-based regional oximeter that interconnects with regional oximetry sensors so as to derive regional oximetry parameters and communicate those parameters to a patient monitor;

FIGS. 2A-B are perspective views of an internal-connector regional oximetry pod and an external-connector regional oximetry pod, respectively;

FIG. 3 is a cross-sectional view of a regional oximetry sensor attached to a tissue site, illustrating corresponding near-field and far-field emitter-to-detector optical paths;

FIG. 4 is a general block diagram of a regional oximetry pod housing a regional oximetry analog board, digital board and signal processor;

FIG. 5 is a general block diagram of regional oximetry signal processing;

FIGS. 6A-D are top perspective, bottom perspective, sensor connector and monitor connector views, respectively, of an internal-connector regional oximetry pod;

FIGS. 7A-D are top perspective, bottom perspective, detailed sensor connector and detailed monitor connector views, respectively, of an external-connector regional oximetry pod;

FIG. 8 is a detailed block diagram of the emitter drive for dual, regional oximetry sensors;

FIG. 9 is a detailed block diagram of the detector interface for dual regional oximetry sensors;

FIG. 10 is a regional oximetry monitor display that provides user I/O showing placement of up to four sensors on a patient; and

FIG. 11 is a regional oximetry parameter display for up to four regional oximetry sensors;

FIGS. 12A-E are various exploded views of an internal-connector regional oximetry pod;

FIGS. 13A-D are side, back, back perspective and exploded views, respectively, of a dual sensor connector for an internal-connector pod;

FIGS. 14A-C are front, front perspective and folded front perspective views, respectively, of an internal-connector flex-circuit assembly for an internal-connector pod; and

FIGS. 15A-C are various exploded views of an external-connector regional oximetry pod.

### DETAILED DESCRIPTION

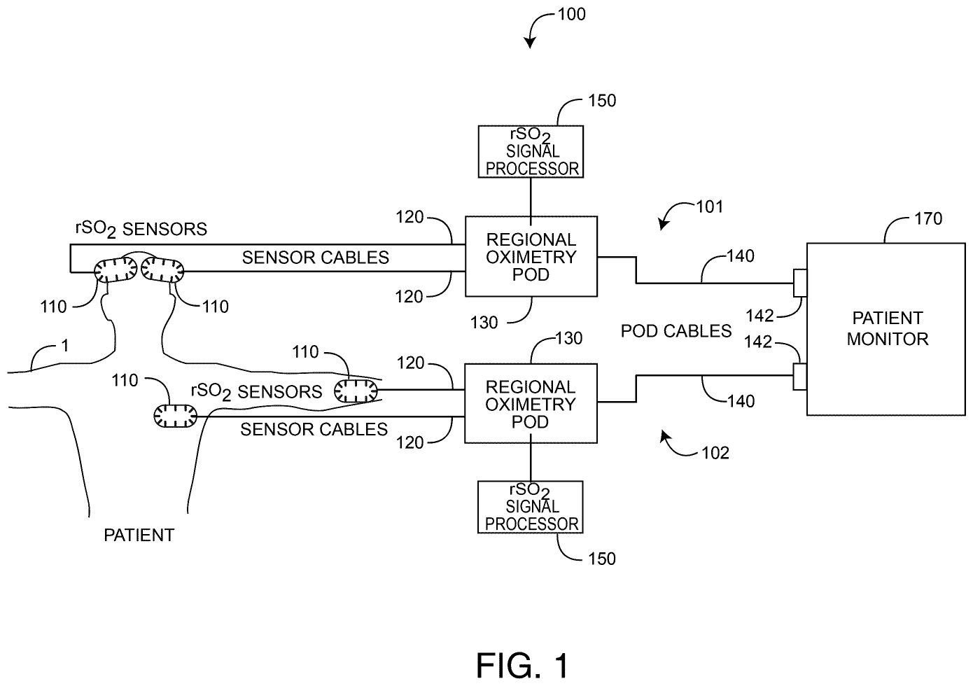

FIG. 1 generally illustrates a pod-based regional oximeter 100 including pod assemblies 101, 102 each communicating with an array of regional oximetry sensors 110 via sensor cables 120. The sensors 110 are attached to various patient 1 locations. One or two regional oximetry pods 130 and a corresponding number of pod cables 140 advantageously provide communications between the sensors 110 and a patient monitor 170. Regional oximetry (rSO₂) signal processors 150 housed in each of the pods 130 perform the algorithmic processing normally associated with patient monitors and/or corresponding monitor plug-ins so as to derive various regional oximetry parameters. The pods 130 communicate these parameters to the patient monitor 170 for display and analysis by medical staff. Further, in an embodiment, each pod 130 utilizes USB communication protocols and connectors 142 to easily integrate with a third party monitor 170. A monitor 170 may range from a relatively “dumb” display device to a relatively “intelligent” multi-parameter patient monitor so as to display physiological parameters indicative of health and wellness.

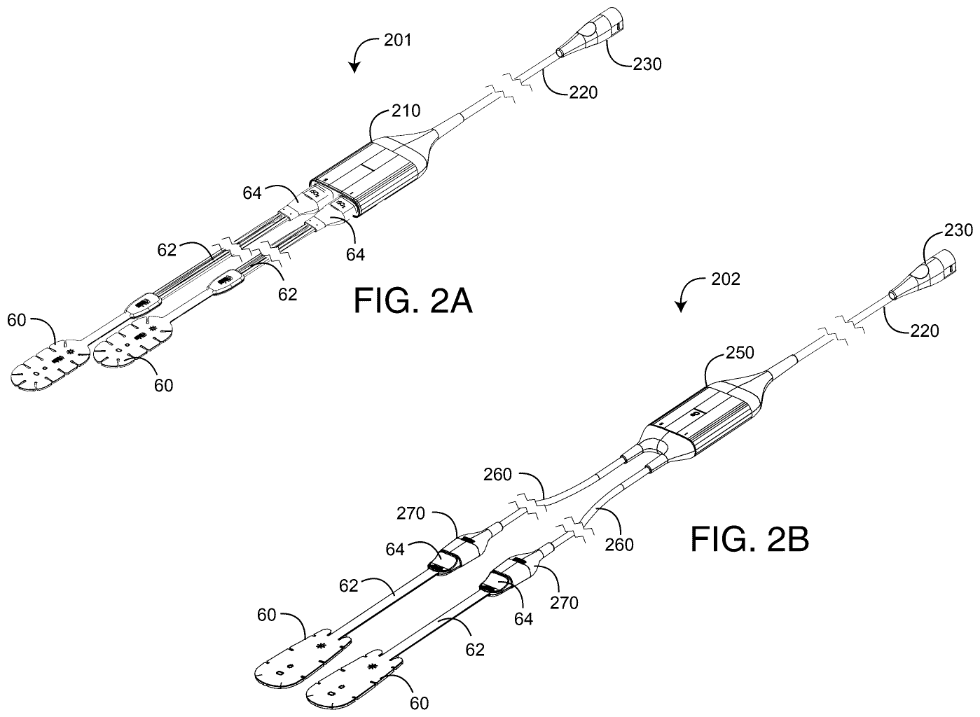

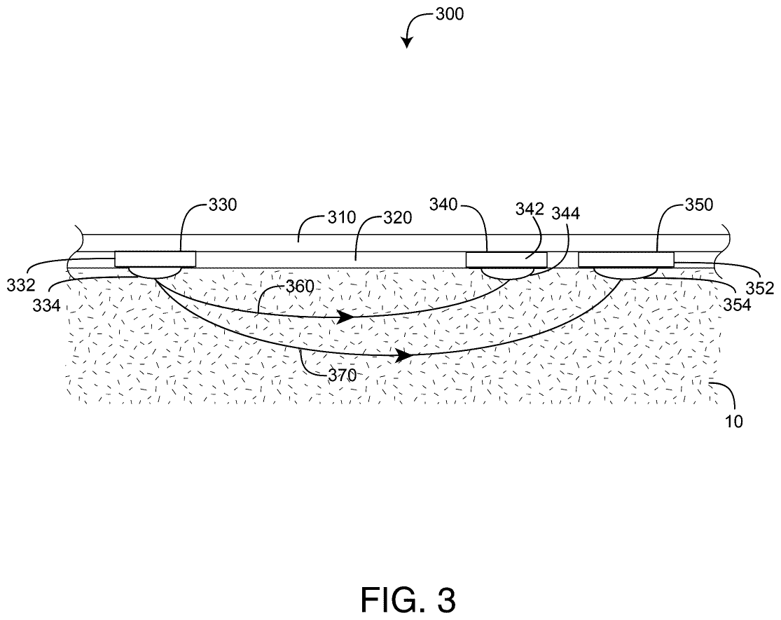

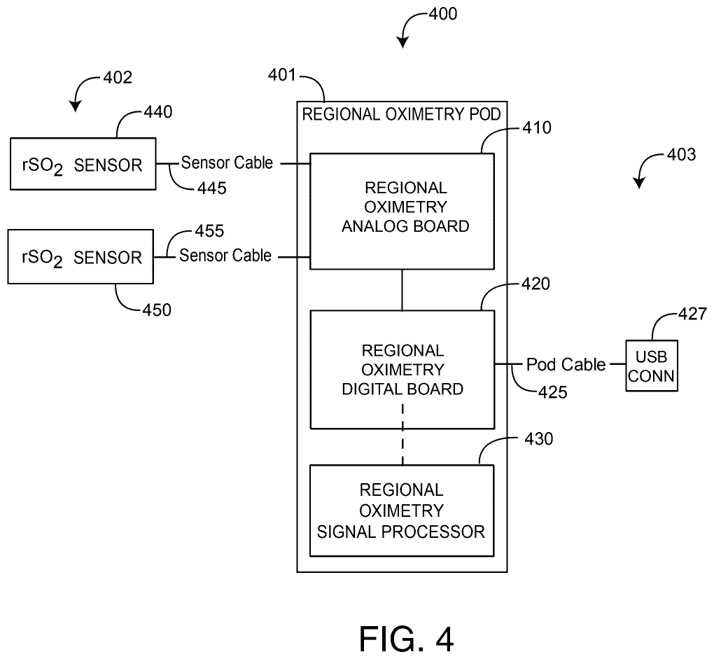

FIGS. 2A-B illustrate an internal-connector regional oximetry pod 201 (FIG. 2A) and an external-connector regional oximetry pod 202 (FIG. 2B). As shown in FIG. 2A, in the internal-connector embodiment 201, pod sockets (not visible) are recessed into the pod housing 210. RSO₂ sensors 60 have sensor cables 62 extending between the sensors 60 and sensor plugs 64. The sensor plugs 64 insert into the pod sockets so as communicate sensor signals between the sensors 60 and pod analog and digital boards (not visible) within the pod housing 210. Pod boards derive regional oximetry parameters, which are communicated to a monitor 170 (FIG. 1) via a monitor cable 220 and a corresponding USB connector 230. Pod boards are described with respect to FIG. 4, below. Sensor optics and corresponding sensor signals are described with respect to FIG. 3, below.

As shown in FIG. 2B, in the external-connector embodiment 202, pod cables 260 extend from the pod housing 250, providing external pod sockets 270. Sensor plugs 64 insert into the external pod sockets 270 so as communicate sensor signals between the sensors 60 and the analog and digital boards within the pod housing 250. As generally described above and in further detail below, pod boards 410, 420 (FIG. 4) derive regional oximetry parameters from the sensor signals, and the parameters are communicated to a monitor 170 (FIG. 1) via the monitor cable 220 and corresponding USB connector 230.

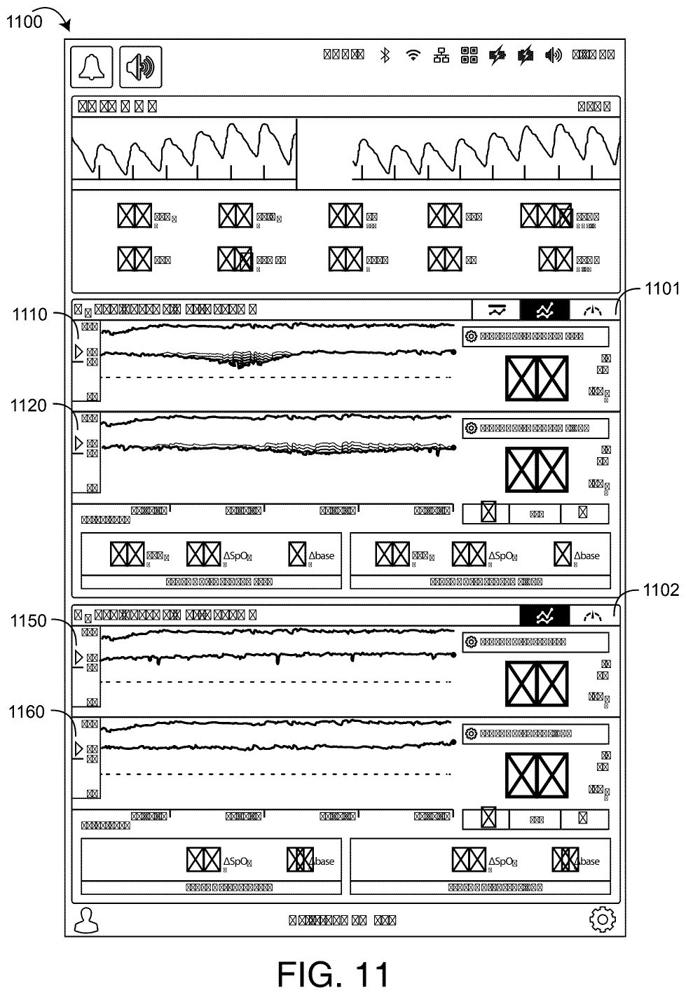

FIG. 3 illustrates a regional oximetry sensor 300 attached to a tissue site 10 so as to generate near-field 360 and far-field 370 emitter-to-detector optical paths through the tissue site 10. The resulting detector signals are processed so as to calculate and display oxygen saturation (SpO₂), delta oxygen saturation (ASpO₂) and regional oxygen saturation (rSO₂), as shown in FIG. 11, below. The regional oximetry sensor 300 has a flex circuit layer 310, a tape layer 320, an emitter 330, a near-field detector 340 and a far-field detector 350. The emitter 330 and detectors 340, 350 are mechanically and electrically connected to the flex circuit 310. The tape layer 320 is disposed over and adheres to the flex circuit 310. Further, the tape layer 320 attaches the sensor 300 to the skin 10 surface.

As shown in FIG. 3, the emitter 330 has a substrate 332 mechanically and electrically connected to the flex circuit 310 and a lens 334 that extends from the tape layer 320. Similarly, each detector 340, 350 has a substrate 342, 352 and each has a lens 344, 354 that extends from the tape layer. In this manner, the lenses 334, 344, 354 press against the skin 10, advantageously maximizing the optical transmission and reception of the emitter 330 and detectors 340, 350.

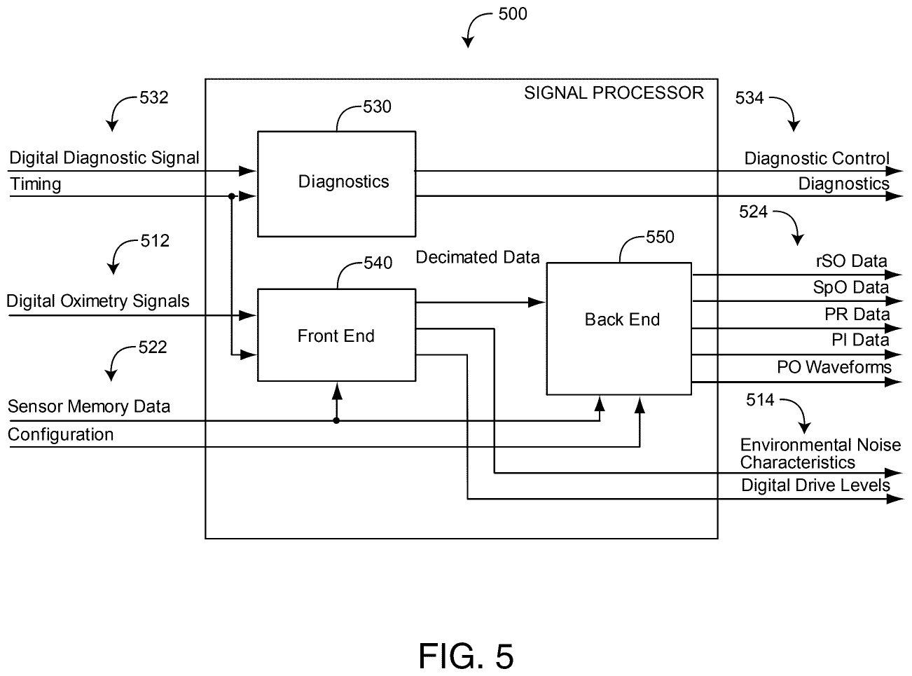

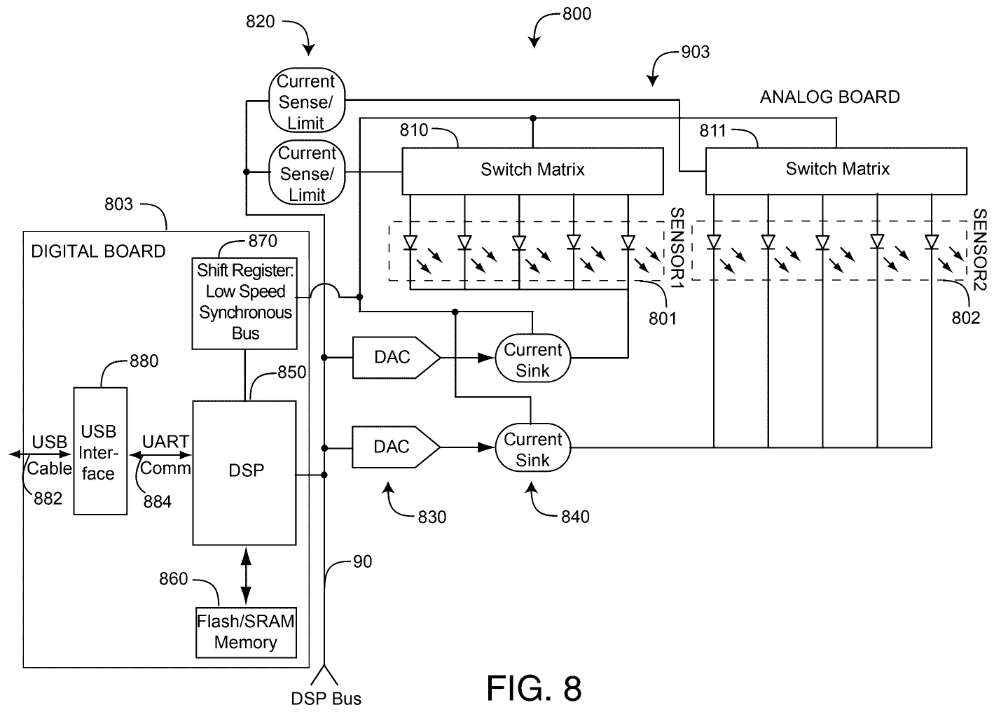

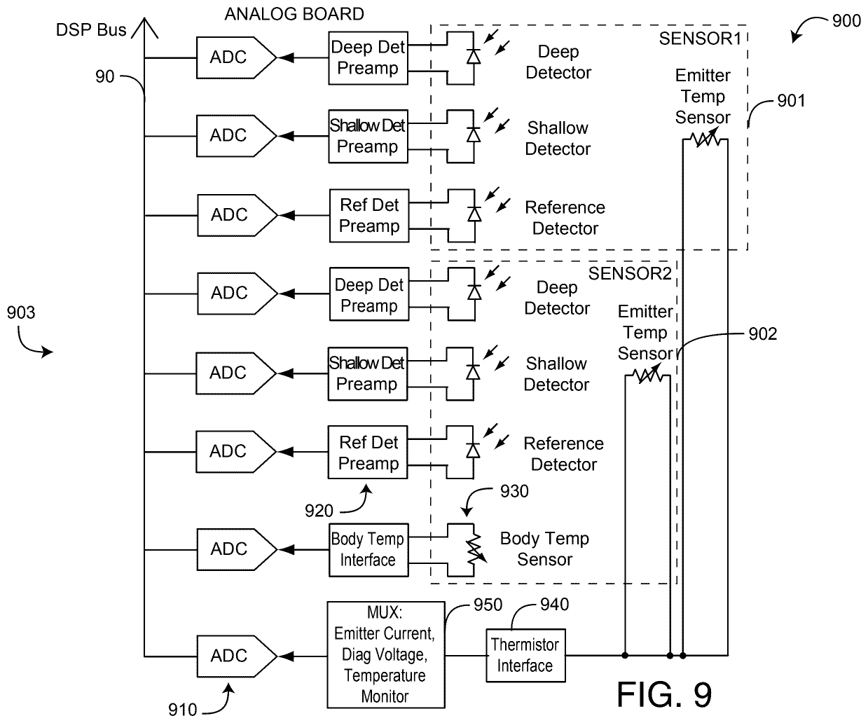

FIG. 4 generally illustrates a regional oximetry pod 401 that houses a regional oximetry analog board 410 and a regional oximetry digital board 420. A regional oximetry signal processor 430 executes on a digital signal processor (DSP) residing on the digital board 420. The regional oximetry signal processor 430 is described with respect to FIG. 5, below. The regional oximetry analog board 410 and digital board 420 are described in detail with respect to FIGS. 8-9, below.

As shown in FIG. 4, on the patient side 402, the regional oximetry analog board 410 communicates with one or more regional oximetry (rSO₂) sensors 440, 450 via one or more sensor cables 445, 455. On the caregiver side 403, a pod cable 425 has a USB connector 427 so as to provide a standard interface between the digital board 420 and a monitor 170 (FIG. 1).

Also shown in FIG. 4, the analog board 410 and the digital board 420 enable the pod 401 itself to perform the sensor communications and signal processing functions of a conventional patient monitor. This advantageously allows pod-derived regional oximetry parameters to be displayed on a variety of monitors ranging from simple display devices to complex multiple parameter patient monitoring systems via the simple USB interface 427.

FIG. 5 generally illustrates a regional oximetry signal processor 500 having a front-end signal processor 540, a back-end signal processor 550 and diagnostics 530. The front end 540 controls LED modulation, detector demodulation and data decimation. The back-end 550 computes sensor parameters from the decimated data. The diagnostics 530 analyze data corresponding to various diagnostic voltages within or external to the digital board so as to verify system integrity.

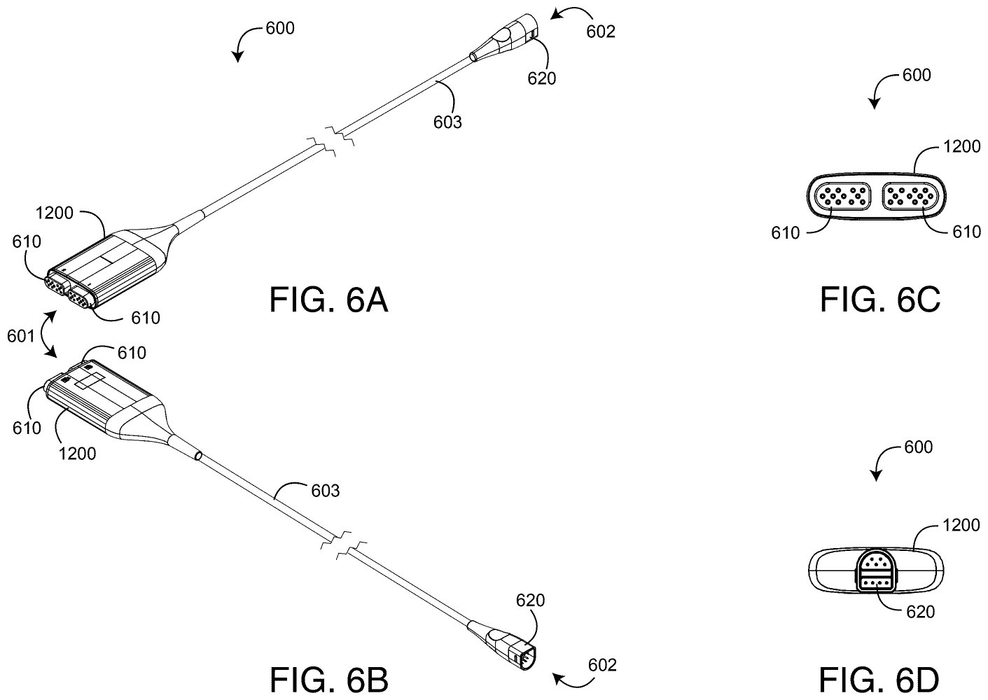

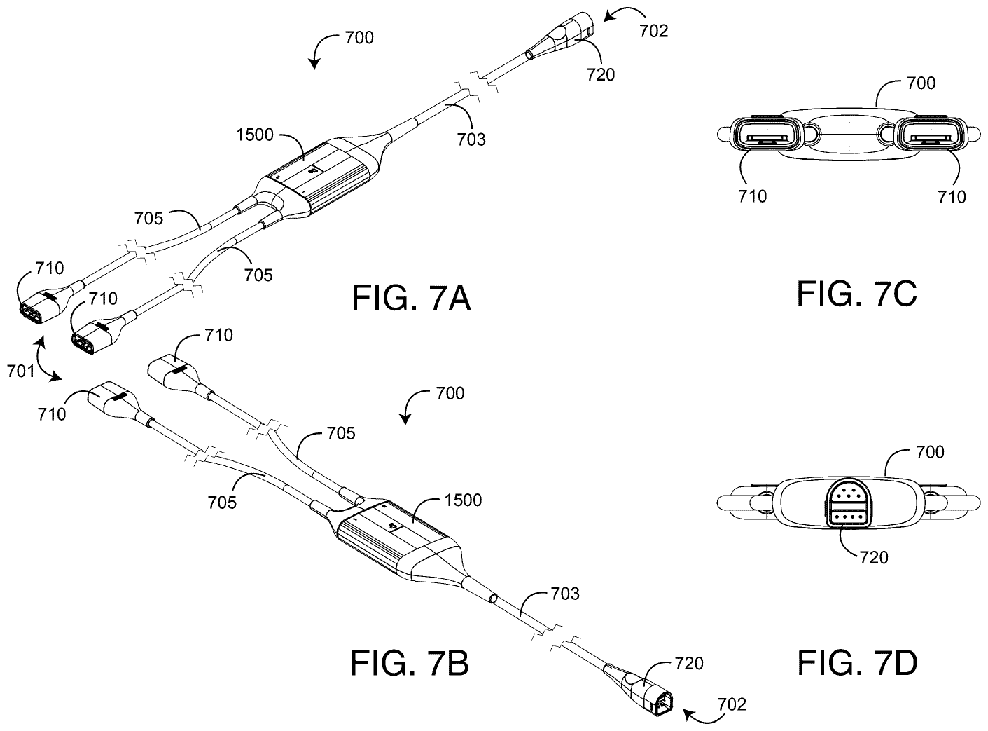

FIGS. 6-7 generally illustrate regional oximetry pod 600, 700 embodiments, each having a pod end 601, 701; a monitor end 602, 702 and an interconnecting pod cable 603, 703. The pod end 601, 701 has dual sensor connectors 610, 710. The monitor end 602, 702 has a monitor connector 620, 720. In a particular embodiment, the monitor connector 620, 720 is a USB connector.

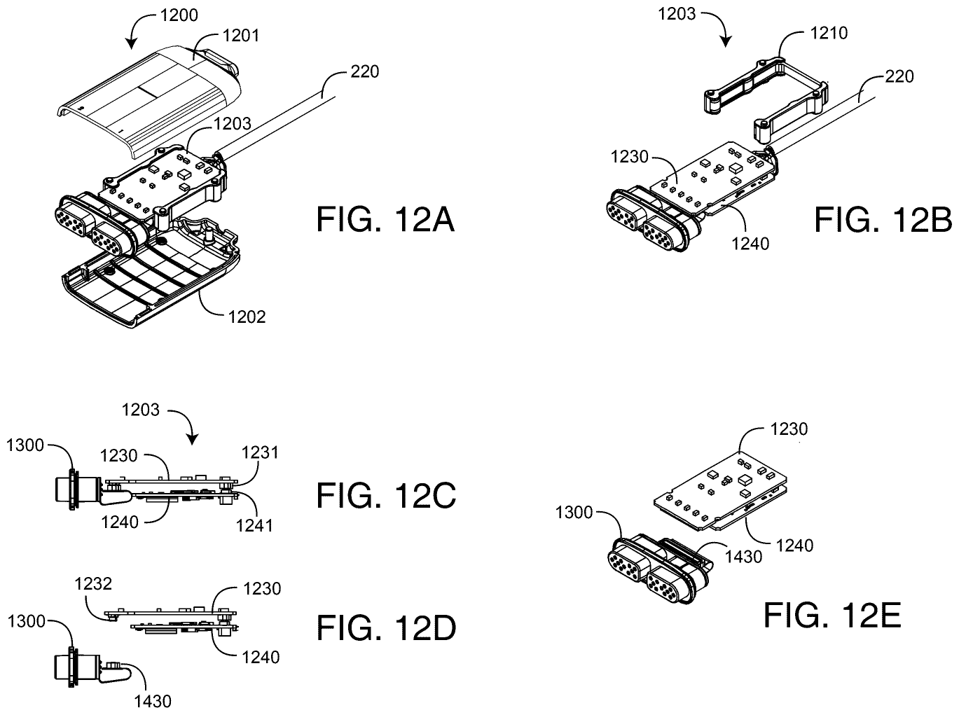

As shown in FIGS. 6A-D, in an internal sensor connector embodiment 600, the sensor connectors 610 are integrated within the pod housing 1200. Advantageously, this configuration provides a relatively compact sensor/monitor interconnection having sensor connectors 610, a monitor connector 620 and an interconnecting pod cable 603. The pod 1200 internals, including the housed portion of the sensor connectors 610, are described in detail with respect to FIGS. 12-14, below.

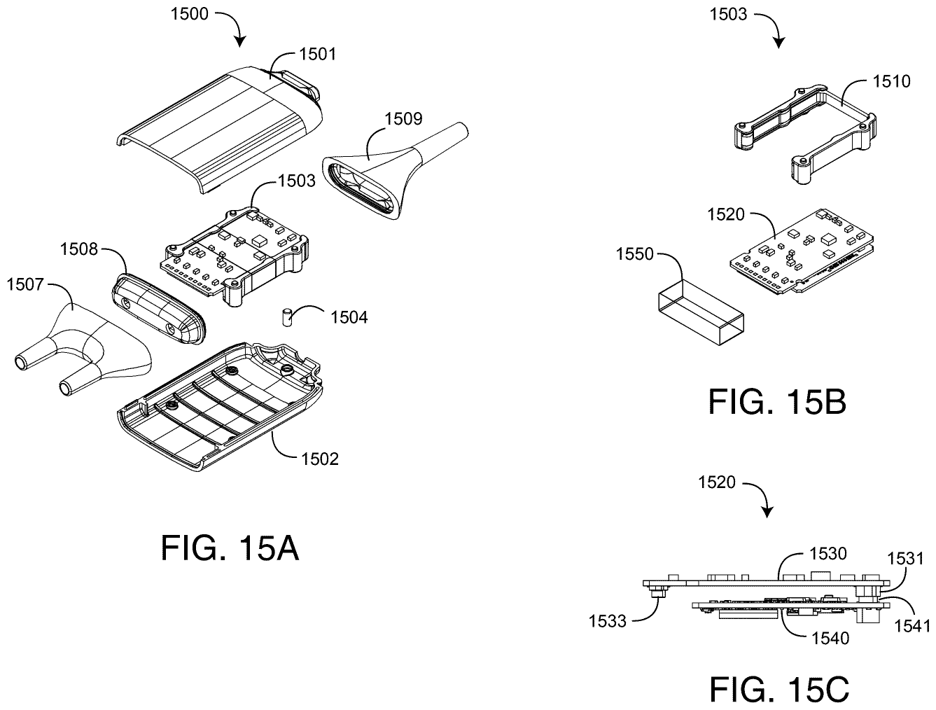

As shown in FIGS. 7A-D, in an external sensor connector embodiment 700, sensor connector cables 705 extend from the pod housing 1500. Advantageously, by removing the dual sensor connectors from within the pod housing 1500, the pod internal complexity is reduced, which reduces manufacturing costs and increases pod reliability. The pod 1500 internals are described in detail with respect to FIG. 15, below.

FIGS. 8-9 illustrate a regional oximetry signal processor embodiment 800, 900 having a digital board 803 (FIG. 8) and an analog board 903 (FIGS. 8-9) in communications with up to two regional oximetry sensors 801, 802 (FIG. 8); 901, 902 (FIG. 9). The digital board 803 (FIG. 8) has a DSP 850 in communications with an external monitor via a USB cable 882 and corresponding UART communications 884. The DSP 850 is also in communications with the sensors 801-802,901-902 via DACs 830 and ADCs 910 on the analog board 903.

As shown in FIGS. 8-9, sensor emitters 801, 802 are driven from the analog board 903 under the control of the digital board DSP 850 via a shift register 870. Each regional sensor 801-802, 901-902 has a shallow detector and a deep detector. Further, each sensor 801-802, 901-902 may have a reference detector and an emitter temperature sensor. In a cerebral regional oximetry embodiment, the sensor(s) may have a body temperature sensor 930 and corresponding analog board ADC 910 interface.

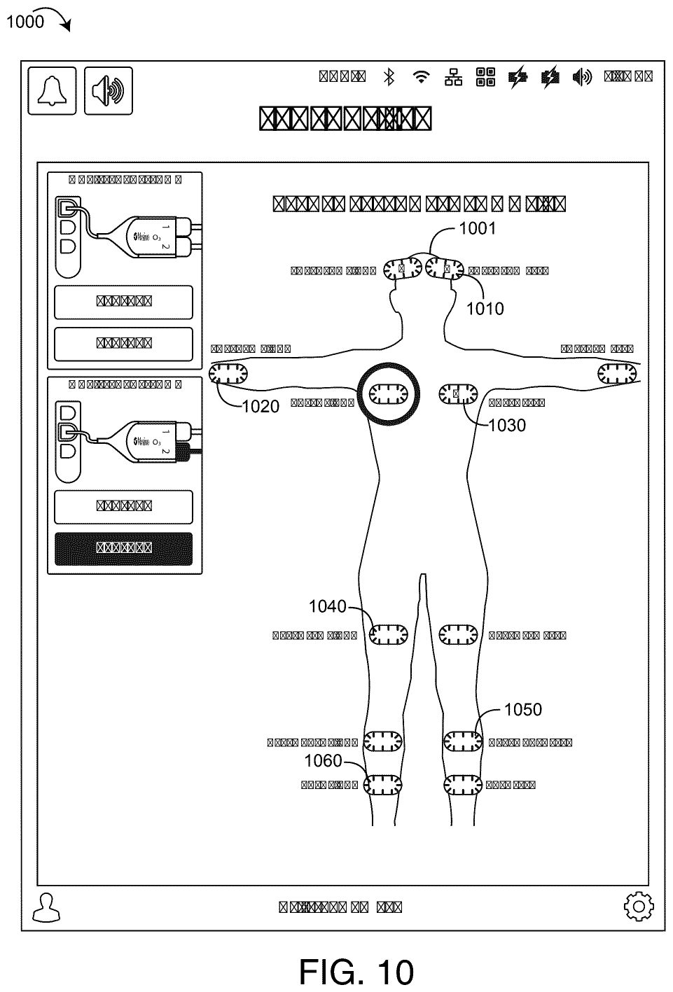

FIG. 10 illustrates a user I/O display 1000 for indicating the placement of up to four sensors on a patient. An adult form 1001 is generated on the display. Between one and four sensor sites can be designated on the adult form 1001, including left and right forehead 1010, forearm 1020, chest 1030, upper leg 1040, upper calf 1050 and right calf 1060 sites. Accordingly, between one and four sensors 110 (FIG. 1) can be located on these sites. A monitor in communication with these sensors then displays between one and four corresponding regional oximetry graphs and readouts, as described with respect to FIG. 11, below.

FIG. 11 illustrates a regional oximetry parameter display 1100 embodiment for accommodating up to four regional oximetry sensor inputs. In this particular example, a first two sensor display 1101 is enabled for monitoring a forehead left site 1110 and a forehead right site 1120. A second two sensor display 1102 is enabled for monitoring a chest left site 1150 and a chest right site 1160.

FIGS. 12A-E further illustrate a regional oximetry pod 1200 embodiment. As shown in FIG. 12A, the pod 1200 has a top shell 1201, a bottom shell 1202, a pod assembly 1203 enclosed between the shells 1201, 1202 and a cable 1241 extending from the pod assembly 1203 through a bend relief (not shown). As shown in FIG. 12B, an analog board 1230 and a digital board 1240 are seated within a frame 1210.

As shown in FIGS. 12C-E, an analog board 1230 is plugged into a dual sensor connector assembly 1300. In particular, an analog board plug 1232 is inserted into a flex circuit assembly socket 1430. With this arrangement, sensor connectors 64 (FIG. 2A) have electrical continuity with the analog board 1230 and the (USB) cable 220 has electrical continuity with the digital board 1240, as described above with respect to FIG. 4.

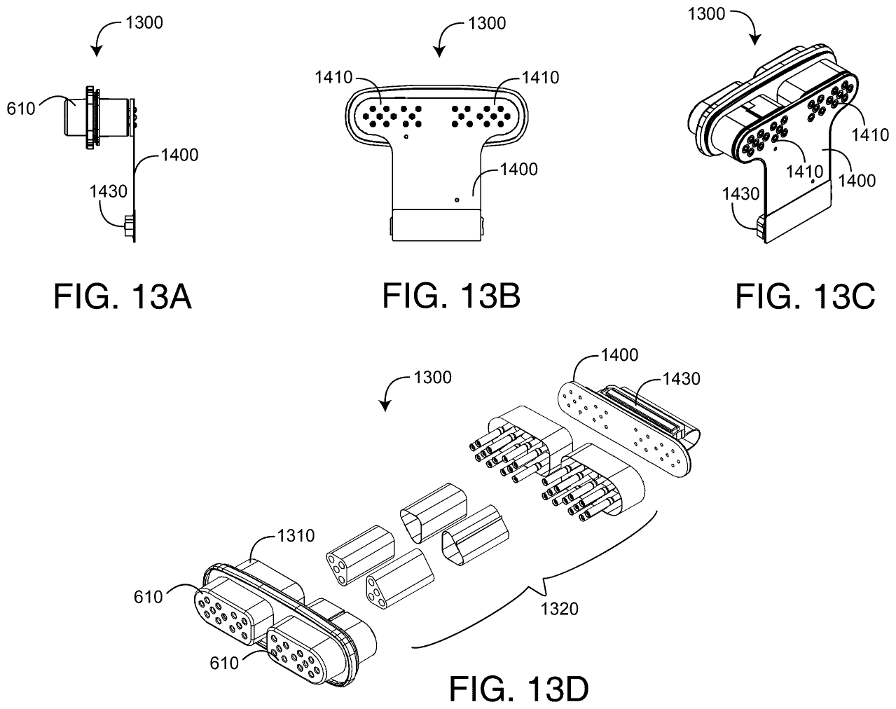

FIGS. 13A-D illustrate a dual sensor connector assembly 1300 that provides communications between the analog board 1230 (FIGS. 12A-E) and the dual sensor connectors 610. The dual sensor connector assembly 1300 has a socket block 1310, a contact assembly 1320 and a flex-circuit assembly 1400. The socket block 1310 retains the contact assembly 1320 so as to form the dual sensor connectors 610. The flex-circuit assembly 1400 provides a socket connector 1430 that mechanically receives analog board plug 1232 (FIG. 12D) and electrically connects the analog board sensor inputs to the sensor connectors 610. In this manner, the analog board 1230 (FIGS. 12A-E) receives sensor signals for signal processing, such as filtering and analog-to-digital conversion.

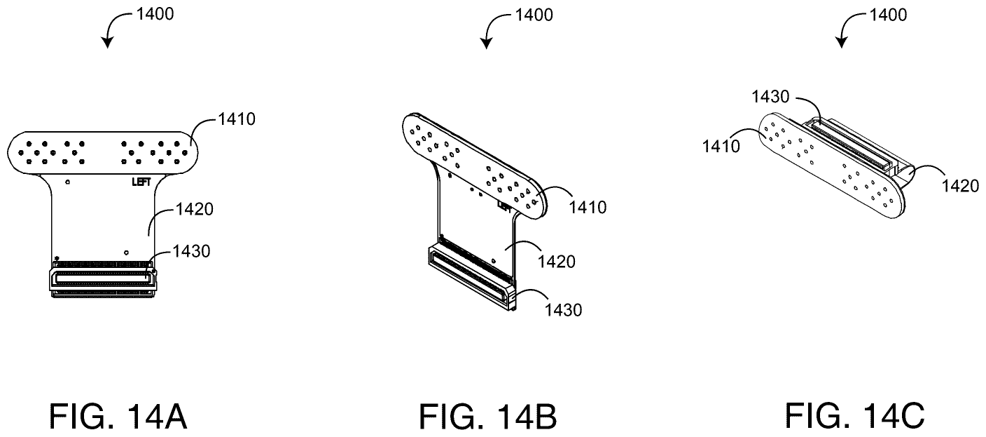

FIGS. 14A-C illustrate a connector flex-circuit assembly 1400 having flex circuit contacts 1410, a flex cable 1420 and a flex circuit socket 1430. The contacts 1410 receive the sensor connector pins 1320 (FIG. 13D), which are soldered in place. When installing the flex-circuit assembly 1400 within a pod 1200 (FIGS. 12A-E) the flex cable 1420 folds into a U-shape (FIG. 14C) so as to expose the flex circuit socket 1430 (FIG. 12D) to the analog board plug 1232 (FIG. 12D), which is then inserted into the socket 1430 (FIG. 12D).

FIGS. 15A-C illustrate an external-connector regional oximetry pod housing 1500 having an upper pod shell 1501 and a lower pod shell 1502 that enclose a board assembly 1503. The board assembly 1503 has a board frame 1510, a signal processing assembly 1520 and a wrap 1550. The board frame 1510 and wrap 1550 mechanically stabilize the signal processing assembly 1520.

As shown in FIGS. 15A-C, the signal processing assembly 1520 has an analog board 1530 and a digital board 1540 as described with respect to FIG. 4, above. The analog board 1530 and a digital board 1540 mechanically and electrically interconnect at board connectors 1531, 1541. A sensor cable 705 (FIGS. 7A-B) threads through an outer sensor cable boot 1507 and an inner sensor cable boot 1508 so as to mechanically and electrically interconnect with an analog board sensor cable connector 1533 (FIG. 15C).

A regional oximetry pod has been disclosed in detail in connection with various embodiments. These embodiments are disclosed by way of examples only and are not to limit the scope of the claims that follow. One of ordinary skill in art will appreciate many variations and modifications.

1. (canceled)

2. A regional oximetry system comprising:
a first regional oximetry sensor comprising:
a sensor body configured to be secured to a subject's forehead;
at least one emitter operably positioned by the sensor body and configured to emit light towards skin of the subject's forehead;
at least one detector operably positioned by the sensor body and configured to detect light reflected from the subject's skin and generate one or more signals based on the detected light; and
a cable connected to the sensor body;
a second regional oximetry sensor comprising:
a sensor body configured to be secured to the subject's forehead;
at least one emitter operably positioned by the sensor body of the second regional oximetry sensor and configured to emit light towards the subject's skin;
at least one detector operably positioned by the sensor body of the second regional oximetry sensor and configured to detect light reflected from the subject's skin and generate one or more signals based on the detected light; and
a cable connected to the sensor body of the second regional oximetry sensor;
a cable adapter for receiving and processing said one or more signals generated by the at least one detector of the first regional oximetry sensor and the at least one detector of the second regional oximetry sensor, the cable adapter comprising:
a housing comprising an interior, a first end, and a second end opposite the first end;
a first connector extending outward from the first end of the housing and configured to removably connect to the cable of the first regional oximetry sensor;
a second connector extending outward from the first end of the housing and configured to removably connect to the cable of the second regional oximetry sensor;
a processor positioned within the interior of the housing and configured to determine one or more physiological parameters of the subject based on said one or more signals; and
a cable extending outward from the second end of the housing; and
a monitoring device configured to removably connect to the cable and receive the processed one or more signals from the cable adapter, the monitoring device further configured to display information relating to said one or more physiological parameters.

3. The regional oximetry system of claim 2, wherein said one or more physiological parameters comprises regional oxygen saturation (rSO₂).

4. The regional oximetry system of claim 2, wherein the cable adapter further comprises:
an analog board positioned within the interior of the housing, the analog board configured to receive and digitize said one or more signals received from the first and second regional oximetry sensors; and
a digital board positioned within the interior of the housing, the digital board comprising said processor.

5. The regional oximetry system of claim 2, wherein:
the at least one detector of the first regional oximetry sensor comprises a near-field detector and a far-field detector spaced from the near-field detector, the near-field detector of the first regional oximetry sensor positioned closer to the at least one emitter of the first regional oximetry sensor than the far-field detector of the first regional oximetry sensor;
the at least one detector of the second regional oximetry sensor comprises a near-field detector and a far-field detector spaced from the near-field detector of the second regional oximetry sensor, the near-field detector of the second regional oximetry sensor positioned closer to the at least one emitter of the second regional oximetry sensor than the far-field detector of the second regional oximetry sensor.

6. A regional oximetry system comprising:
a first regional oximetry sensor configured to be secured to skin of a subject, the first regional oximetry sensor comprising:
at least one emitter configured to emit light towards the subject's skin;
at least one detector configured to detect light reflected from the subject's skin and generate one or more signals based on the detected light; and
a second regional oximetry sensor configured to be secured to the subject's skin, the second regional oximetry sensor comprising:
at least one emitter configured to emit light towards the subject's skin;
at least one detector configured to detect light reflected from the subject's skin and generate one or more signals based on the detected light; and
a cable adapter for receiving and processing said one or more signals generated by the at least one detector of the first regional oximetry sensor and the at least one detector of the second regional oximetry sensor, the cable adapter comprising:
a housing comprising an interior, a first end, and a second end;
a first connector arranged at the first end of the housing and configured to removably connect to the first regional oximetry sensor;
a second connector arranged at the first end of the housing and configured to removably connect to the second regional oximetry sensor;
a processor positioned within the interior of the housing and configured to determine one or more physiological parameters of the subject based on said one or more signals; and
wherein the second end of the housing is configured to be connected to a monitoring device via a cable to allow said one or more physiological parameters to be transmitted to the monitoring device.

7. The regional oximetry system of claim 6, wherein the cable adapter further comprises said cable, and wherein said cable extends outward from the second end of the housing.

8. The regional oximetry system of claim 6, wherein said one or more physiological parameters comprises regional oxygen saturation (rSO₂).

9. The regional oximetry system of claim 6, wherein the cable adapter further comprises:
an analog board positioned within the interior of the housing, the analog board configured to receive and digitize said one or more signals received from the first and second regional oximetry sensors; and
a digital board positioned within the interior of the housing, the digital board comprising said processor.

10. The regional oximetry system of claim 6, wherein:
the at least one detector of the first regional oximetry sensor comprises a near-field detector and a far-field detector spaced from the near-field detector, the near-field detector of the first regional oximetry sensor positioned closer to the at least one emitter of the first regional oximetry sensor than the far-field detector of the first regional oximetry sensor;
the at least one detector of the second regional oximetry sensor comprises a near-field detector and a far-field detector spaced from the near-field detector of the second regional oximetry sensor, the near-field detector of the second regional oximetry sensor positioned closer to the at least one emitter of the second regional oximetry sensor than the far-field detector of the second regional oximetry sensor.

11. The regional oximetry system of claim 6, further comprising said monitoring device, wherein said monitoring device is configured to display information relating to said one or more physiological parameters.

12. The regional oximetry system of claim 6, wherein each of the first connector and the second connector is partially arranged within the interior of the housing.

13. A regional oximetry system comprising:
a cable adapter comprising:
a housing comprising an interior, a first end, and a second end;
a first connector arranged at the first end and configured to removably connect to a first regional oximetry sensor;
a second connector arranged at the first end and configured to removably connect to a second regional oximetry sensor; and
a processor positioned within the interior of the housing and configured to determine one or more physiological parameters of a subject based on one or more signals received from the first and second regional oximetry sensors;
wherein the second end of the housing is configured to be connected to a monitoring device via a cable to allow said one or more physiological parameters to be transmitted to the monitoring device.

14. The regional oximetry system of claim 13, wherein said one or more physiological parameters comprises regional oxygen saturation (rSO₂).

15. The regional oximetry system of claim 13, wherein the cable adapter further comprises:
an analog board positioned within the interior of the housing, the analog board configured to receive and digitize said one or more signals; and
a digital board positioned within the interior of the housing, the digital board comprising said processor.

16. The regional oximetry system of claim 13, further comprising said monitoring device, wherein said monitoring device is configured to display information relating to said one or more physiological parameters.

17. The regional oximetry system of claim 16, wherein the cable adapter further comprises said cable, and wherein said cable extends outward from the second end of the housing.

18. The regional oximetry system of claim 13, wherein each of the first connector and the second connector is partially arranged within the interior.

19. The regional oximetry system of claim 13, further comprising said first and second regional oximetry sensors.

19. The regional oximetry system of claim 19, wherein each of the first and second regional oximetry sensors is configured to be secured to skin of the subject and comprises:
at least one emitter configured to emit light towards the subject's skin;
at least one detector configured to detect light reflected from the subject's skin and generate said one or more signals based on the detected light.

21. The regional oximetry system of claim 20, wherein:
the at least one detector of the first regional oximetry sensor comprises a near-field detector and a far-field detector spaced from the near-field detector, the near-field detector of the first regional oximetry sensor positioned closer to the at least one emitter of the first regional oximetry sensor than the far-field detector of the first regional oximetry sensor;
the at least one detector of the second regional oximetry sensor comprises a near-field detector and a far-field detector spaced from the near-field detector of the second regional oximetry sensor, the near-field detector of the second regional oximetry sensor positioned closer to the at least one emitter of the second regional oximetry sensor than the far-field detector of the second regional oximetry sensor.
